(19) United States
(12) Patent Application Publication
(10) Pub. No.: US 2022/0189623 A1
(43) Pub. Date: Jun. 16, 2022

(54) SYSTEMS AND METHODS OF GUIDED INFORMATION INTAKE

(71) Applicant: STATE FARM MUTUAL AUTOMOBILE INSURANCE COMPANY, Bloomington, IL (US)

(72) Inventors: Edward W. Breitweiser, Bloomington, IL (US); Jennifer L. Crawford, Normal, IL (US); Brian Steigerwald, Bloomington, IL (US); Danielle Malan, Downs, IL (US)

(21) Appl. No.: 17/550,482

(22) Filed: Dec. 14, 2021

Related U.S. Application Data
(60) Provisional application No. 63/125,653, filed on Dec. 15, 2020.

(51) Int. Cl. G16H 40/63; G10L 15/22; G10L 15/18

(52) U.S. Cl. CPC G16H 40/63; G10L 15/18; G10L 15/22

(57) ABSTRACT

Systems and methods relating to collecting guided user data generating recommendations are disclosed, with particular reference to collecting user data related to the well-being of a user. Such systems and methods include obtaining well-being data associated with a user and determining that a triggering event has occurred based at least upon the well-being data collected. A user log may be generated by iteratively presenting a plurality of information prompts to the user. In generating the user log, the systems and methods may apply a conversational artificial intelligence model, receive user responses to prompts, and add data entries to the user log. A user action recommendation based upon the data entries may be generated and presented to the user.

### CROSS-REFERENCE TO RELATED APPLICATIONS

This application claims the benefit of U.S. Provisional Application Ser. No. 63/125,653 filed Dec. 15, 2020, which is incorporated herein by reference.

### TECHNICAL FIELD

The present disclosure generally relates to systems and methods for guided user data collection and recommendation generation.

### BACKGROUND

The average individual may have a number of tasks of varying importance vying for a limited amount of attention. Unfortunately, well-being often falls to the wayside in the face of other, more pressing concerns. Physical and mental well-being, social well-being, financial well-being, and other forms of well-being may often go ignored, particularly where traditional methods for promoting well-being are no longer available.

Further, even when an individual is aware of the importance of properly maintaining well-being, the individual may not have the background necessary to craft a proper method for the requisite maintenance. This may lead to ineffectual attempts that, at best, lack the efficiency that a properly-designed plan may allow or, at worst, become outright dangerous to the individual in question. This may be especially true in the world today, in which there are many pressures on the well-being of the average individual—physical, social, financial, and mental—with little in the way of conventional respite.

Without taking individualized needs into account, any instructions that may be provided quickly and/or on a large scale may often also be either inefficient or outright incorrect. Individualized data must be provided to form individualized instruction. However, because of the various objectives vying for attention, an individual may oftentimes forget or fail to see the need for particular updates to be made. Thus, efforts to promote well-being may be inefficient and/or incorrect due to a lack of relevant, individualized data as well as a lack of proper, individualized instruction. Conventional techniques may have other drawbacks as well.

### SUMMARY

The present embodiments relate to, inter alia, guided user data collection and recommendation generation to promote user well-being. In one aspect, a computer-implemented method for guided user data collection and recommendation generation may be provided. The computer-implemented method may include, via one or more local or remote processors, servers, sensors, and/or transceivers: (i) obtaining well-being data associated with a user; (ii) determining occurrence of a triggering event associated with the user based upon the well-being data; (iii) generating a user log containing a plurality of user data entries by iteratively presenting a plurality of information prompts to the user in response to the occurrence of the triggering event by, for each of the plurality of information prompts: (a) applying a conversational artificial intelligence model to the well-being data and any user data entries in the user log to identify the respective information prompt; (b) presenting the information prompt to the user via a user interface; (c) receiving a user response to the information prompt via the user interface; (d) adding a user data entry indicative of the user response to the user log; and/or (e) determining whether to present an additional information prompt to the user; (iv) generating a user action recommendation based upon the user data entries of the user log; and/or (v) presenting the user action recommendation to the user via the user interface. The computer-implemented method may include additional, fewer, or alternate actions, including those discussed elsewhere herein.

Systems or computer-readable media storing instructions for implementing all or part of the methods described above may also be provided in some aspects. Systems for implementing such methods may include one or more of the following: a client computing device of a user, a remote server, one or more sensors, one or more communication modules configured to communicate wirelessly via radio links, radio frequency links, and/or wireless communication channels, and/or one or more program memories coupled to one or more processors of any such computing devices or servers. Such program memories may store instructions to cause the one or more processors to implement part or all of the method described above.

### BRIEF DESCRIPTION OF DRAWINGS

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

The Figures described below depict various aspects of the applications, methods, and systems disclosed herein. It should be understood that each Figure depicts an embodiment of a particular aspect of the disclosed applications, systems and methods, and that each of the Figures is intended to accord with one or more possible embodiments thereof. Furthermore, wherever possible, the following description refers to the reference numerals included in the following Figures, in which features depicted in multiple Figures are designated with consistent reference numerals.

FIG. 1 illustrates a block diagram of an exemplary well-being application system for monitoring and improving user well-being.

FIG. 2A illustrates a diagram of an exemplary guided user data collection and recommendation generation application, in which a user has an exemplary exchange with a conversational artificial intelligence model triggered by geolocation data and/or temporal data.

FIG. 2B illustrates a diagram of an exemplary guided user data collection and recommendation generation application, in which a user has an exemplary exchange with a conversational artificial intelligence model triggered by browsing data and/or purchase history data.

FIG. 2C illustrates a diagram of an exemplary guided user data collection and recommendation generation application, in which a user has an exemplary exchange with a conversational artificial intelligence model triggered by social media data.

FIG. 2D illustrates a diagram of an exemplary guided user data collection and recommendation generation application, in which a well-being journal entry is populated by user data, trigger events, and well-being recommendations.

FIG. 3A illustrates a flow diagram of an exemplary guided user data collection and recommendation generation method to indicate a typical flow for generating a user log, generating a user action recommendation, and presenting the user action recommendation to a user.

FIG. 3B illustrates a flow diagram of an exemplary computer-implemented method for generating a user log containing a plurality of user data entries.

FIG. 4 illustrates a flow diagram of an exemplary computer-implemented method for generating a user log containing a plurality of user data entries to indicate an exemplary flow for identifying and presenting an information prompt to a user.

FIG. 5 illustrates a flow diagram of an exemplary computer-implemented method for generating a user log containing a plurality of user data entries to indicate an exemplary flow for receiving a vocalized answer from a user and updating a ranking based upon at least an analysis of the vocalized answer.

FIG. 6 illustrates a flow diagram of an exemplary computer-implemented method for generating a user log containing a plurality of user data entries to indicate an exemplary flow using well-being data in determining a triggering event has occurred and using collected medical data, trend data, and metadata in generating a user action recommendation.

FIG. 7 illustrates a flow diagram of an exemplary computer-implemented method for generating a user log containing a plurality of user data entries to indicate an exemplary flow for determining whether to present an additional prompt based upon at least well-being data.

FIG. 8 illustrates a flow diagram of an exemplary computer-implemented method for training a conversational artificial intelligence 800 using gathered user data.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

The average individual is not always aware of her own well-being and/or of the steps required to keep well-being maintained. While generalities tend to be widely known, such as the importance of staying hydrated, the specifics are much more obscure. Similarly, even being aware of the specifics of important well-being tasks, such as staying hydrated, does not help the average busy individual going about her day.

In order to provide accurate and timely information to an individual and/or optimize well-being as much as possible for an individual, the techniques disclosed herein generally describe collecting data related to the well-being of a user, and using the well-being data to generate a user log tracking the well-being of the user. From the information stored in the user log, the application disclosed herein generates a user action recommendation.

The user log may be generated by iteratively presenting a plurality of information prompts to the user and applying a conversational artificial intelligence model to the well-being data and any user data entries in the user log. The use of a conversational artificial intelligence model allows the program to more accurately determine relevant information prompts and user action recommendations. A more accurate determination similarly leads to a faster overall process.

The user action recommendations may be single time recommendations, such as a recommendation to schedule an appointment with a physician. The user action recommendations may additionally or alternatively be a plurality of recommendations made in a consistent manner so as to act as a reminder for the user. For example, a reminder to drink an appropriate amount of water, or a daily reminder to take medicine.

Well-being data, as defined herein, refers to both classical physical health data (e.g., a user has a fever or a broken limb), but also to mental health data. Further, well-being data also refers to more than just health-related well-being. Social well-being, financial well-being, and legal well-being—amongst other forms—are all contemplated and discussed herein.

### Exemplary Well-Being Application System

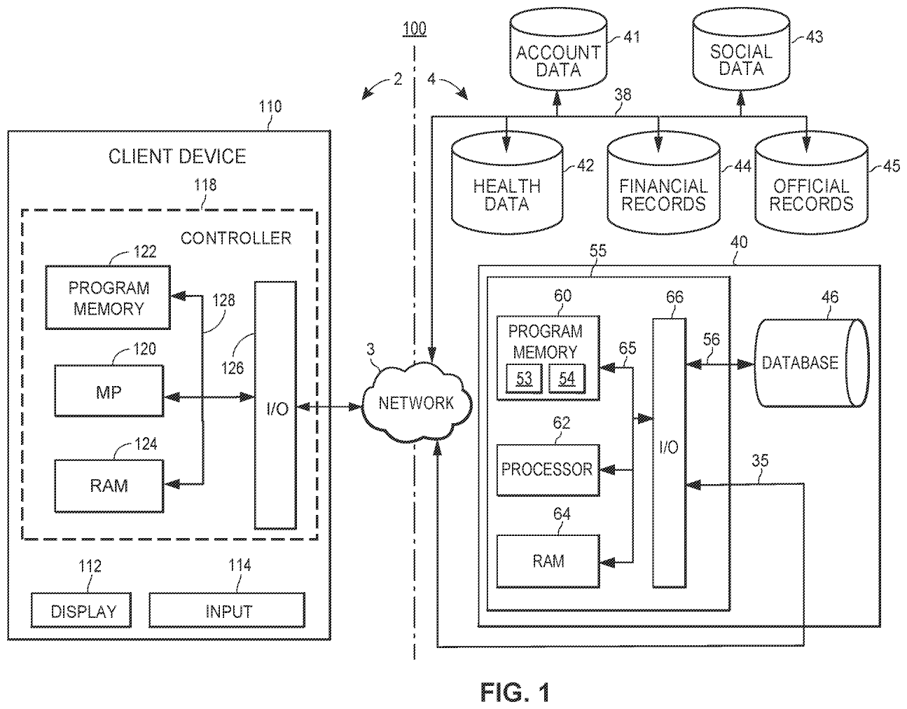

FIG. 1 illustrates a block diagram of an exemplary well-being application system 100 on which the exemplary computer-implemented methods described herein may be implemented to monitor and improve user well-being. The high-level architecture may include both hardware and software applications, as well as various data communications channels for communicating data between the various hardware and software components. The well-being application system 100 may be roughly divided into front-end components 2 and back-end components 4. The front-end components 2 may be associated with users of a well-being application to monitor, manage, and/or enhance their well-being related to physical health, mental health, financial condition, document organization, social connection, community involvement, or other aspects of well-being. The back-end components 4 may include hardware and software components implementing aspects of such well-being applications, as well as internal or external data sources associated with user well-being data.

In some embodiments of the system 100, the front-end components 2 may communicate with the back-end components 4 via a network 3. One or more client devices 110 associated with users of the well-being application may communicate with the back-end components 4 via the network 3 to receive data from and provide data to back-end components 4 associated with the well-being application. The back-end components 4 may use one or more servers 40 to receive data and data requests from the front-end components 2, process and store received data, access additional data sources, analyze user well-being, provide data to the front-end components 2, and/or perform additional well-being application functions as described herein. The one or more servers 40 may also communicate with other back-end components 4, such as additional data sources 41-45. Some embodiments may include fewer, additional, or alternative components.

The front-end components 2 may be disposed within one or more client devices 110, which may include a desktop computer, notebook computer, netbook computer, tablet computer, or mobile device (e.g., a cellular telephone, smart phone, wearable computer, smart speaker, smart appliance, IoT device, etc.). The client device 110 may include a display 112, an input 114, and a controller 118. In some embodiments, the client device 110 may further include a Global Positioning System (GPS) unit (not shown) to determine a geographical location of the client device 110. The input 114 may include a “soft” keyboard that is displayed on the display 112 of the client device 110, an external hardware keyboard communicating via a wired or a wireless connection (e.g., a Bluetooth keyboard), an external mouse, or any other suitable user-input device. The input 114 may further include a microphone, camera, or other input device capable of receiving information. The controller 118 includes one or more microcontrollers or microprocessors (MP) 120, a program memory 122, a RAM 124, and an I/O circuit 126, all of which may be interconnected via an address/data bus 128. The program memory 122 may include an operating system, a data storage, a plurality of software applications, and/or a plurality of software routines.

The program memory 122 may include software applications, routines, or scripts for implementing communications between the client device 110 and the server 40 or additional data sources 41-45 via the network 3. For example, the program memory 122 may include a web browser program or application, thereby enabling the user to access web sites via the network 3. As another example, the program memory 122 may include a social media application that receives data from and sends data to a social data source 43 via the network 3. The program memory 122 may further store computer-readable instructions for a program or application associated with one or more well-being applications.

In some embodiments, the controller 118 may also include, or otherwise be communicatively connected to, other data storage mechanisms (e.g., one or more hard disk drives, optical storage drives, solid state storage devices, etc.) that reside within the client device 110. It should be appreciated that although FIG. 1 depicts only one microprocessor 120, the controller 118 may include multiple microprocessors 120. Similarly, the memory of the controller 118 may include multiple program memories 122 or multiple RAMs 124. Although the FIG. 1 depicts the I/O circuit 126 as a single block, the I/O circuit 126 may include a number of different types of I/O circuits. The controller 118 may implement the program memories 122 or the RAMs 124 as semiconductor memories, magnetically readable memories, or optically readable memories, for example.

The various computing devices of the front-end components 2 may communicate with the back-end components 4 via wired or wireless connections to the network 3. The network 3 may be a proprietary network, a secure public internet, a virtual private network or some other type of network, such as dedicated access lines, plain ordinary telephone lines, satellite links, cellular data networks, or combinations of these. The network 3 may include one or more radio frequency communication links, such as wireless communication links with client devices 110. The network 3 may also include other wired or wireless communication links with other client devices 110 or other computing devices. Where the network 3 may include the Internet, data communications may take place over the network 3 via an Internet communication protocol.

The back-end components 4 may include one or more servers 40 configured to implement part or all of the processes related to the well-being application described herein. Each server 40 may include one or more computer processors adapted and configured to execute various software applications and components of the well-being application system 100, in addition to other software applications. The server 40 may further include a database 46, which may be adapted to store data related to user well-being for a plurality of users and/or data relating to recommendations or incentives for users. Such data may include data related to user preferences, user conditions, user policies or accounts, user property, user actions, user incentives, user goals, goal progress, user biometric data, or other well-being data relating to a user, as discussed elsewhere herein, part or all of which data may be collected by or uploaded to the server 40 via the network 3. The server 40 may access data stored in the database 46 or external data sources when executing various functions and tasks associated with the computer-implemented methods discussed elsewhere herein.

The server 40 may have a controller 55 that is operatively connected to the database 46 via a link 56. It should be noted that, while not shown, additional databases may be linked to the controller 55 in a known manner. For example, separate databases may be used for various types of information, such as user profiles, user activity data, or well-being data models. Additional data sources 41-45 may be communicatively connected to the server 40 via the network 3, such as databases maintained by third parties or databases associated with other servers 40. The controller 55 may include a program memory 60, a processor 62 (which may be called a microcontroller or a microprocessor), a random-access memory (RAM) 64, and an input/output (I/O) circuit 66, all of which may be interconnected via an address/data bus 65.

It should be appreciated that although only one processor 62 is shown, the controller 55 may include multiple processors 62. Similarly, the memory of the controller 55 may include multiple RAMs 64 and multiple program memories 60. Although the I/O circuit 66 is shown as a single block, it should be appreciated that the I/O circuit 66 may include a number of different types of I/O circuits. The RAM 64 and program memories 60 may be implemented as semiconductor memories, magnetically readable memories, or optically readable memories, for example. The controller 55 may also be operatively connected to the network 3 via a link 35.

The server 40 may further include a number of software applications stored in a program memory 60. The various software applications on the server 40 may include one or more software applications for monitoring, storing, evaluating, generating, and tracking user well-being data or recommendations. Such software applications may include a well-being evaluation module 53 configured to generate user well-being metrics or identify user well-being conditions, as well as artificial intelligence models 54 trained and used for generating user well-being recommendations, as discussed further below. The various software applications may be executed on the same computer processor or on different computer processors.

The back-end components 4 may further include one or more additional data sources 41-45 providing information relating to aspects of user well-being. These additional data sources 41-45 may be configured to communicate with the server 40 through the network 3 via a link 38. The additional data sources 41-45 may include an account data source 41, a health data source 42, a social data source 43, a financial records data source 44, and/or an official records data source 45. Information regarding various aspects of users' physical, mental, social, or financial health may be stored in databases associated with the various additional data sources 41-45, which data may be accessed as part of the computer-implemented methods described herein. In some embodiments, additional or alternative data sources may be accessed to obtain further information relevant to user well-being.

The account data source 41 may maintain user account data for a plurality of user accounts associated with users of client devices 110. In some embodiments, such user account data may include user profiles for such users, which may be associated with various services, such as telecommunications services, financial services, insurance policies, or well-being services. For example, user account data may include information regarding insurance policies, bank accounts, and investment accounts associated with a user.

The health data source 42 may maintain user health data associated with a plurality of users, such as biometrics data from wearable computing devices or electronic medical records. Such user health data may be used to monitor aspects of user physical and mental health. In some embodiments, the user health data may relate to individuals associated with a user of a well-being application, such as young children or elderly parents of the user.

The social data source 43 may maintain user social data associated with a plurality of users, such as users of social media platforms. Such social data may be used to identify life events based upon user profile information updates, user posts, or posts referencing a user. In some embodiments, user posts or metadata regarding user posts may also be analyzed to determine user social connections or mental well-being (e.g., stress levels, connectedness, depression, loss, etc.), which may include performing user sentiment analysis.

The financial records data source 44 may maintain user financial records, such as banking or investment records. In some embodiments, financial records may include credit-related records maintained by credit rating agencies, such as revolving accounts, loans, assets, or contractual agreements (e.g., leases, utility, or other services). Such financial records may be used to determine user financial well-being or to generate recommendations regarding improvements in the user's current financial condition or planning for future events (e.g., collecting, organizing, or reviewing financial documents).

The official records data source 45 may maintain official records regarding a user, such as records maintained by various governmental agencies. Such official records may include user property records, licensure records, birth/death records, benefits records, or other official records associated with a user's well-being. In some embodiments, official records may include notices published in newspapers of records or other reliable non-governmental sources.

Although the well-being application system 100 is shown to include one or a limited number of the various front-end components 2 and of the back-end components 4, it should be understood that different numbers of any or each of these components may be utilized in various embodiments. Furthermore, the database storage or processing performed by the one or more servers 40 and/or additional data sources 41-45 may be distributed among a plurality of components in an arrangement known as “cloud computing.” This configuration may provide various advantages, such as enabling near real-time uploads and downloads of information, as well as providing additional computing resources needed to handle the monitoring, modeling, evaluation, and/or recommendation tasks described herein. This may in turn support a thin-client embodiment of some computing devices of the front-end components 2, such as some client devices 110.

### Exemplary Guided User Data Collection and Recommendation Generation Methods

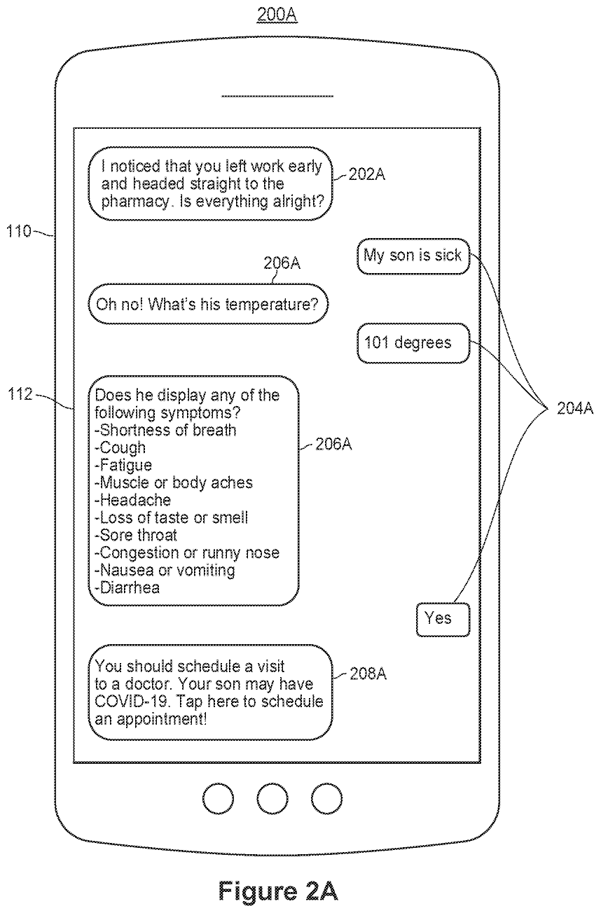

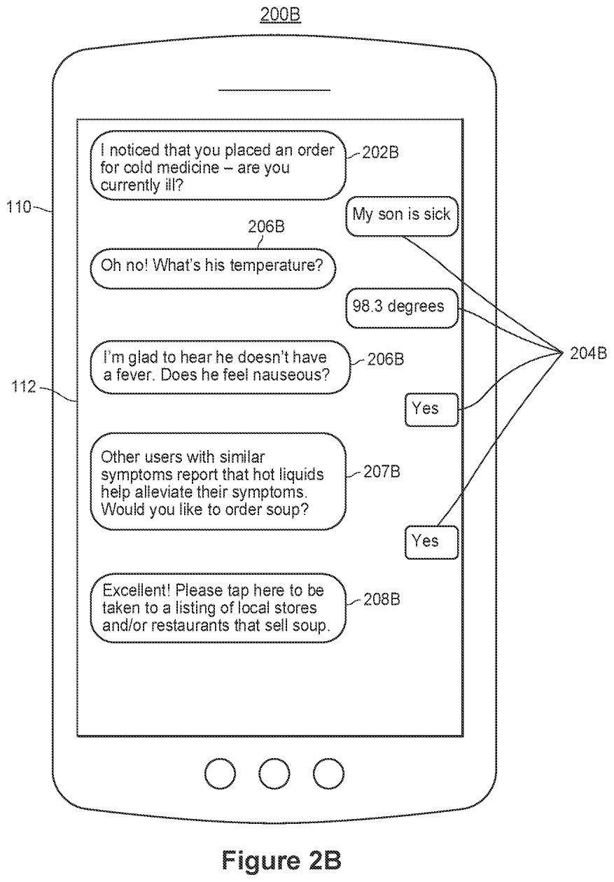

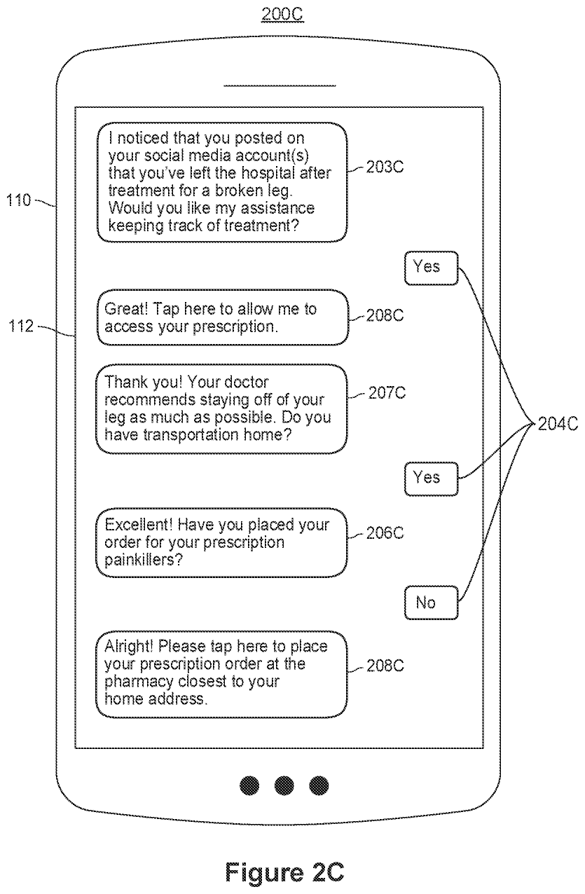

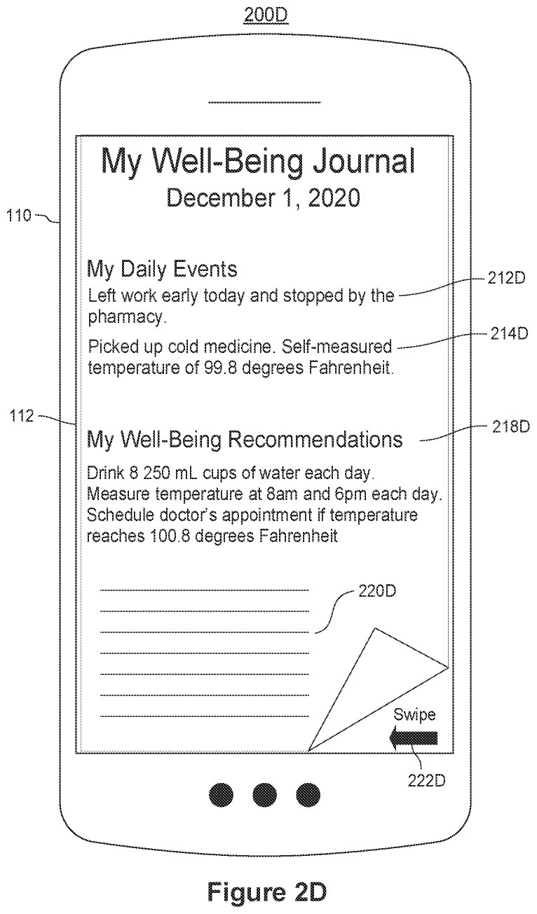

FIGS. 2A-D illustrate diagrams of an exemplary guided user data collection and recommendation generation application 200A-D implemented on a client device 110 and presented via a display 112 of the client device 110. The depictions of guided user data collection and recommendation generation application 200A-D are exemplary only, and other implementations may include additional, fewer, or alternative actions and/or objects. Various parts of the guided user data collection and recommendation generation application 200A-D may be implemented by or performed using the various front-end components 2 and back-end components 4, which may communicate via the network 3, as described above. In some embodiments, the guided user data collection and recommendation generation application 200A-D may be implemented by one or more servers of a cloud computing service (e.g., one or more servers 40 configured to implement a single guided user data collection and recommendation generation platform).

Where numbering remains consistent for objects in FIGS. 2A-D, the objects in question behave similarly and have similar qualities. As such, the objects are described only once each, unless at least one of FIGS. 2A-D treats the object in question differently.

Object 202A depicts a message sent to the client device 110 by the conversational artificial intelligence model 54, which determines that a triggering event takes place before displaying an information prompt to the user by way of the display 112 of the client device 110. In some embodiments, the message includes the information prompt and a notification of the triggering event (e.g., “I noticed that you left work early and headed straight to the pharmacy,” as the triggering event; “Is everything alright?” as the information prompt). In other embodiments, the message includes the information prompt, a notification of the triggering event, and/or a user action recommendation (e.g., “I noticed that your test for COVID-19 was positive. If you experience any of the following symptoms, visit the ER immediately.”). In still other embodiments, the message includes information about the triggering event (e.g., information on common causes of a symptom, information on effects of dehydration, information on potential changes in insurance policy for which the user may be eligible, etc.).

In some embodiments, the triggering event is based upon well-being data gathered from the user. In further embodiments, the well-being data includes at least one of (i) social media data, (ii) browsing data, (iii) biometric data, (iv) smart device data, (v) geolocation data, or (vi) user-input data. In some implementations, the client device 110 gathers the well-being data from the user. In other implementations, the server 40 gathers the well-being data from additional data sources such as account data 41, health data 42, social data 43, financial records 44, and/or official records 45. Similarly, the server 40 may gather the well-being data from a database 46.

Objects 204A depict user responses to information prompts. In some embodiments, a user response 204A is analyzed by the server 40 using the conversational artificial intelligence model 54 and natural language processing (NLP) techniques. In some implementations, the user response 204A includes extraneous information that the conversational artificial intelligence model 54 ignores. As such, the conversational artificial intelligence model 54 may analyze user responses 204A by identifying and using keywords present in the user response 204A.

In various embodiments, the user response 204A may be vocalized, typed, or otherwise presented via a combination of the two (e.g., using speech-to-text features, vocalizing part of the answer and typing specifics, etc.).

Objects 206A depict additional information prompts. In some embodiments, the conversational artificial intelligence model 54 provides additional information prompts 206A until the conversational artificial intelligence model 54 determines that one appropriate user action recommendation remains. In other embodiments, the conversational artificial intelligence model 54 provides additional information prompts 206A until a predetermined number of user action recommendations remain. In further embodiments, the conversational artificial intelligence model 54 provides additional information prompts 206A until a predetermined number of additional information prompts 206A have been provided. The predetermined number of user action recommendations and/or additional information prompts 206A may be determined by the conversational artificial intelligence model 54, by the user, or by configuration settings of the program.

Object 208A depicts a user action recommendation. In some embodiments, the user may directly interact with the user action recommendation 208A to perform the recommended action (e.g., by tapping the message to schedule a medical appointment). In further embodiments, the server 40 may have sufficient permissions such that the user action recommendation 208A is a notification to the user of action taken by the conversational artificial intelligence model 54. For example, the user may grant permission for the conversational artificial intelligence model 54 to schedule appointments with reference to a calendar feature. In such an instance, the conversational artificial intelligence model 54 may determine that the user is showing dangerous symptoms and automatically schedule a medical appointment for an available time slot. The conversational artificial intelligence model 54 may then present the appointment information to the user via the display 112 of the client device 110, along with an option to cancel or modify the appointment.

Object 207B depicts a notification of trend data associated with successful user action recommendations combined with an additional information prompt. In some embodiments, the trend data may be associated with user data from the user in question. In further embodiments, the trend data may be gathered from other users of the application 200D. In still further embodiments, the trend data may be gathered from one or more third party sources (e.g., health websites, blogs, medical publications, social media, etc.).

Object 203C depicts a notification of a triggering event and a request for permission for the conversational artificial intelligence model 54 to begin generating a user log. In various embodiments, the request for permission may be in addition to or in place of blanket permission from the user. In various implementations, the request for permission may be presented to the user once, daily, once per individual triggering event, once per unique triggering event, or in any other increment as determined by the user and/or the conversational artificial intelligence model 54.

Object 208C depicts a user action recommendation. In some embodiments, the user action recommendation 208C may follow a request for permission 203C. The user action recommendation 208C may not end a particular instance of a journal. For example, the user action recommendation 208C may lead to additional trend data that establishes relevance of particular issues, such as is depicted in object 207C. In further embodiments, the user action recommendation 208C may be a triggering event. In such instances, the application 200 may log the user action recommendation 208C separately and/or associate the user action recommendation 208C with the previous triggering event.

Object 212D depicts a log of triggering events taking place throughout a day. In some embodiments, the triggering events may be separated based on relevance. For example, triggering events related to one another may be grouped together and identified as such (e.g., leaving work early and stopping by the pharmacy being two related triggering events). Similarly, triggering events that are not directly related but have similar user action recommendations may be grouped together (e.g., general dehydration as a triggering event being grouped with purchasing cold medicine as a triggering event due to both having the user action recommendations related to drinking water). In further embodiments, the user may group the triggering events manually.

Object 214D depicts a log of user responses entered into the well-being journal application 200D throughout a day. In some embodiments, the conversational artificial intelligence model 54 automatically records the user responses in a log 214D. In further embodiments, the conversational artificial intelligence model 54 prompts the user by way of the display 112 of the user device 110 to provide an accounting of user responses. In various implementations, the user responses may be kept together with and/or separate from the relevant triggering events and/or information prompts.

Object 218D depicts a log of user action recommendations suggested by the conversational artificial intelligence model 54 throughout a day. Depending upon the embodiment, the conversational artificial intelligence model 54 may automatically record user action recommendations in the log 218D. The conversational artificial intelligence model 54 may also only record user action recommendations in the log 218D that the user chooses. In some embodiments, the user may log user action recommendations 218D manually.

Object 220D depicts a notetaking area in which a user can make notes on user action recommendations, triggering events, and/or information prompts. In some embodiments, the conversational artificial intelligence model 54 may read and use the notes in notetaking area 220D as user data when making future determinations of triggering events, information prompts, and/or user action recommendations. In further embodiments, the notes in notetaking area 220D are for the user as personal notes.

Object 222D depicts an indication for moving between entries in the well-being journal application 200D. In some embodiments, a user may move between entries in the well-being journal application 200D by swiping with a finger at the denoted location 222D. In other embodiments, a user may move between entries by swiping with a finger anywhere on the display 112. In various other embodiments, the user may move between entries by pressing a button, shaking the client device 110, speaking aloud, and/or performing any other commonly-utilized method in the art of interface navigation.

Though the above descriptions discuss the user providing information to the conversational artificial intelligence 54 via text and the client device 110 providing information to the user visually, other methods may also be applied. For example, a user may speak aloud to the input 114 (e.g., a microphone) of the client device 110 to provide user responses. Similarly, the client device 110 may provide information prompts or data to the user via audio-based means. It would be clear to one skilled in the art to make use of other input and/or presentation methods in carrying out the disclosure. Similarly, it would be clear to one skilled in the art that specified instances of time and/or length should not be limiting to the disclosure. For example, instances of daily activities could be applied to weekly activities instead, depending upon the implementation.

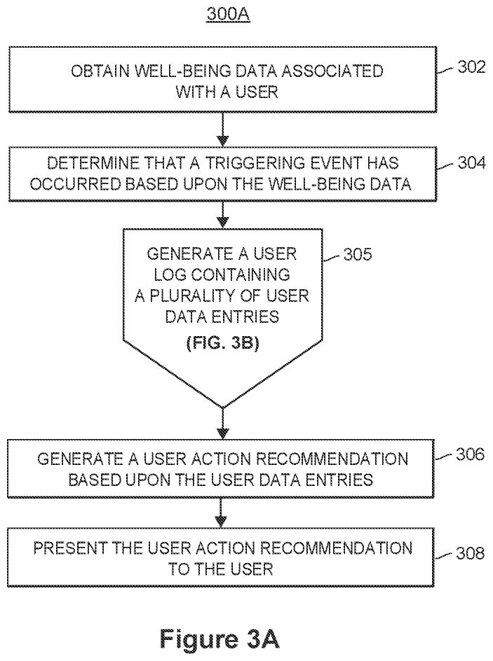

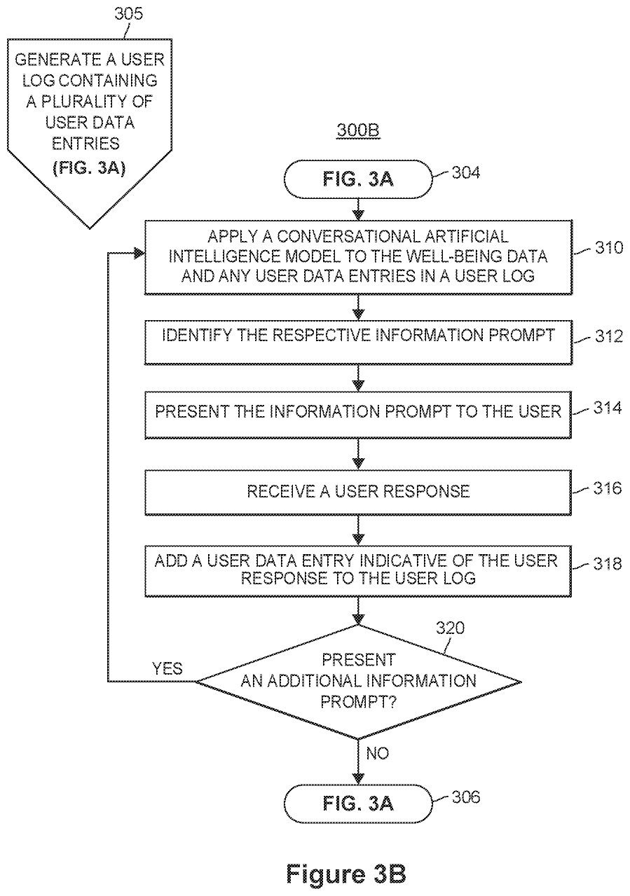

FIG. 3A illustrates a flow diagram of an exemplary computer-implemented guided user data collection and recommendation generation method 300A to indicate a typical flow for generating a user log, generating a user action recommendation, and presenting the user action recommendation to a user. The computer-implemented guided user data collection and recommendation generation method 300A is exemplary only, and other methods may include additional, fewer, or alternative actions.

Various parts of the computer-implemented guided user data collection and recommendation generation method 300A may be implemented by or performed using the various front-end components 2 and back-end components 4, which may communicate via the network 3, as described above. In some embodiments, the computer-implemented guided user data collection and recommendation generation method 300A may be implemented by one or more servers of a cloud computing service (e.g., one or more servers 40 configured to implement a single guided user data collection and recommendation generation platform).

At block 302, the computer-implemented guided user data collection and recommendation generation method 300A may begin by obtaining well-being data associated with a user at a server 40 from client devices 110 associated with a user. The well-being data may include user data regarding physical health, mental health, social data, financial records, official records, account data, geolocation data, temporal data, etc.

At block 304, the server 40 determines that a triggering event has occurred based at least upon the well-being data obtained at block 302. The triggering event may be based upon any or all of the well-being data. For example, the server 40 may make use of geolocation and temporal data to recognize that a user has left the workplace earlier than usual, which may flag to the server 40 that a triggering event has occurred. Physical health data or scheduling data may indicate that a triggering event has not actually occurred despite the change to routine, however, and computer-implemented method 300A may cease.

At block 305, the server 40 generates a user log containing a plurality of user data entries. As explored in more detail in FIG. 3B below, the server may generate this user log via applying a conversational artificial intelligence model 54 to the well-being data and to any user data entries in the user log iteratively. The server 40 may prompt the user with one or more information prompts to identify a proper recommendation. The prompting may be done visually or vocally, and the answer may be received through text or via NLP techniques. In some embodiments, the information prompts may be decided upon via a probabilistic valuation ranking technique. In further embodiments, the conversational artificial intelligence model 54 may be trained using machine learning techniques.

At block 306, the server 40 generates a user action recommendation based at least in part upon the user data entries. In some embodiments, the user action recommendation may be a recommendation for a user to purchase or use a particular type of object associated with well-being (e.g., medicine, healthcare products, food, exercise equipment, etc.). In further embodiments, the user action recommendation may be a recommendation to perform a particular action (e.g., visit a physician, exercise, drink water, etc.). In still further embodiments, the user action recommendation may be a recommendation to provide the server 40 with more information (e.g., take a temperature, stretch an affected area, test the air quality, etc.) along with instructions on how to perform the action in question. The server 40 may also generate a user action recommendation that combines any or all of the above embodiments as well as others not explicitly discussed above.

At block 308, the server 40 presents the user action recommendation to the user. In some embodiments, the server 40 may present the user action recommendation visually via a display 112 of a client device 110 associated with the user. In further embodiments, the server 40 may present the user action recommendation through an audio-based method, such as via speakers of the client device 110. In still further embodiments, the server 40 may present the user action recommendation using some combination of visual and audio-based methods.

FIG. 3B illustrates a flow diagram of an exemplary computer-implemented method for generating a user log containing a plurality of user data entries 300B as part of block 305 of computer-implemented method 300A. As such, the exemplary computer-implemented method 300B occurs after block 304 and before block 306 in FIG. 3A. Any identical numbering between FIGS. 3A and 3B indicate the same action between the two figures. The computer-implemented method for generating a user log containing a plurality of user data entries 300B is exemplary only, and other methods may include additional, fewer, or alternative actions. Various parts of the computer-implemented guided user data collection and recommendation generation method 300B may be implemented by or performed using the various front-end components 2 and back-end components 4, which may communicate via the network 3, as described above. In some embodiments, the computer-implemented guided user data collection and recommendation generation method 300B may be implemented by one or more servers of a cloud computing service (e.g., one or more servers 40 configured to implement a single guided user data collection and recommendation generation platform).

At block 310, the server 40 applies a conversational artificial intelligence model 54 to the well-being data, as well as any user data entries already present in a user log. The conversational artificial intelligence model 54 may be trained by processing large quantities of data via machine learning techniques. In some embodiments, the data may come from the user carrying out computer-implemented method 300B. In further embodiments, the data may come from other users of the application. In still further embodiments, the data may come from a third party source. In some implementations, the conversational artificial intelligence model 54 is trained using deep learning and/or neural network techniques.

At block 312, the server 40 identifies the respective information prompt. In some embodiments, this identification may be based at least in part upon a valuation and ranking system implemented by the conversational artificial intelligence model 54. In further embodiments, this identification may be based at least in part upon well-being data. The well-being data may include user data regarding physical health, mental health, social data, financial records, official records, account data, geolocation data, temporal data, etc.

At block 314, the client device 110 presents the information prompt to the user. In some embodiments, the client device 110 may present the information prompt to the user visually via a display 112. In further embodiments, the client device 110 may present the information prompt to the user through an audio-based method, such as speakers. In still further embodiments, the client device 110 may present the information prompt through some combination of visual and audio-based methods.

At block 316, the client device 110 receives a user response that the client device 110 then transmits to the server 40. In some embodiments, the conversational artificial intelligence model 54 uses NLP techniques to analyze the user response. In further embodiments, the conversational artificial intelligence model 54 compares the user response to past responses by the user to analyze the user response. For example, the conversational artificial intelligence model 54 may determine that the user has a tendency to misspell a particular word. In such instances, the conversational artificial intelligence model 54 may determine the meaning of the word despite the errors. In still further embodiments, the conversational artificial intelligence model 54 compares the user response to responses by other users to analyze the user response. In yet further embodiments, the conversational artificial intelligence model 54 compares the user response to third party data to analyze the user response.

At block 318, the server adds a user data entry to the user log that is indicative of the user response. In some embodiments, the user data entries added to the user log may contain metadata regarding an aspect of the user response associated with a time, a length, a duration, or a delay of the user response.

At block 320, the server 40 determines whether or not to present an additional information prompt. If so, the computer-implemented method loops back to block 310. If not, the computer-implemented method continues on to block 306 of FIG. 3A. In some implementations, the determination may be based at least in part upon well-being data.

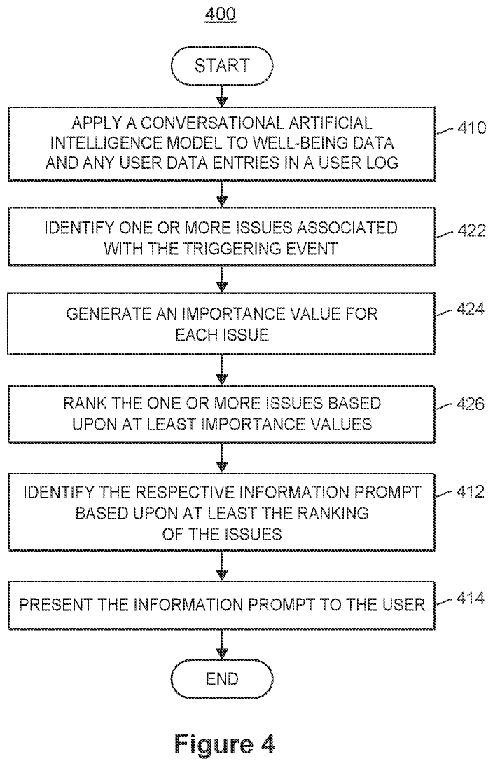

FIG. 4 illustrates a flow diagram of an exemplary computer-implemented method 400 for generating a user log containing a plurality of user data entries to indicate an exemplary flow for identifying and presenting an information prompt to a user. Any similar numbering between FIGS. 3A&B and FIG. 4 indicates a similar action. The computer-implemented method 400 is exemplary only, and other methods may include additional, fewer, or alternative actions, including those discussed elsewhere herein.

Various parts of the computer-implemented guided user data collection and recommendation generation method 400 may be implemented by or performed using the various front-end components 2 and back-end components 4, which may communicate via the network 3, as described above. In some embodiments, the computer-implemented guided user data collection and recommendation generation method 400 may be implemented by one or more servers of a cloud computing service (e.g., one or more servers 40 configured to implement a single guided user data collection and recommendation generation platform).

At block 410, the server 40 applies a conversational artificial intelligence model 54 to the well-being data as well as any user data entries already present in a user log. The conversational artificial intelligence model 54 may be trained by processing large quantities of data via machine learning techniques. In some embodiments, the data may come from the user carrying out computer-implemented method 400. In further embodiments, the data may come from other users of the application. In still further embodiments, the data may come from a third party source. In some implementations, the conversational artificial intelligence model 54 is trained using deep learning and/or neural network techniques.

At block 422, the conversational artificial intelligence model 54 identifies one or more issues associated with the triggering event. In some embodiments, these issues may be potential side effects or symptoms of the triggering condition itself. In further embodiments, these issues may be associated with distant potential effects of the triggering condition. In some implementations, the issues may be identified by comparison to past user data from the same user (e.g., issues that are associated with leaving work early, issues associated with stopping by the pharmacy, issues associated with activating a wearable smart device, etc.). In other implementations, the issues may be identified by comparison to other users and/or third party databases (e.g., what are the most common illnesses historically for the time of year). Generally, the issues may be modified, added to, or subtracted from depending upon at least user responses and/or a determined importance value assigned to an issue.

At block 424, the conversational artificial intelligence model 54 generates an importance value for each issue. The importance value is generated based upon at least a determined probabilistic likelihood of each issue applying to the triggering event. In some embodiments, the likelihood is determined by analyzing past user data of the user. In further embodiments, the likelihood is determined by past medical records of the user (e.g., a family history of diabetes may increase the likelihood of dietary-related issues). In still further embodiments, the likelihood is determined by comparing issues related to the triggering event to the user data of other users and/or third party databases. Generally, the conversational artificial intelligence model 54 may change the importance values as the conversational artificial intelligence model 54 iteratively presents the plurality of information prompts to the user and/or the user responds.

At block 426, the conversational artificial intelligence model 54 ranks the one or more issues based upon at least importance values. The conversational artificial intelligence model 54 may modify the issue ranking as the user responds to the plurality of information prompts.

At block 412, the conversational artificial intelligence model 54 identifies the respective information prompt based upon at least the ranking of the issues. In some embodiments, the conversational artificial intelligence model 54 identifies the respective information prompt as the prompt that has an issue with the highest ranking. In other embodiments, the conversational artificial intelligence model 54 identifies the respective information prompt as the prompt that has the highest average ranking amongst the issues associated with the prompt. In further embodiments, the conversational artificial intelligence model 54 removes issues common to potential prompts when identifying the respective information prompt.

At block 414, the client device 110 presents the information prompt to the user. In some embodiments, the client device 110 may present the information prompt to the user visually via a display 112. In further embodiments, the client device 110 may present the information prompt to the user through an audio-based method, such as speakers. In still further embodiments, the client device 110 may present the information prompt to the user through some combination of visual and audio-based methods.

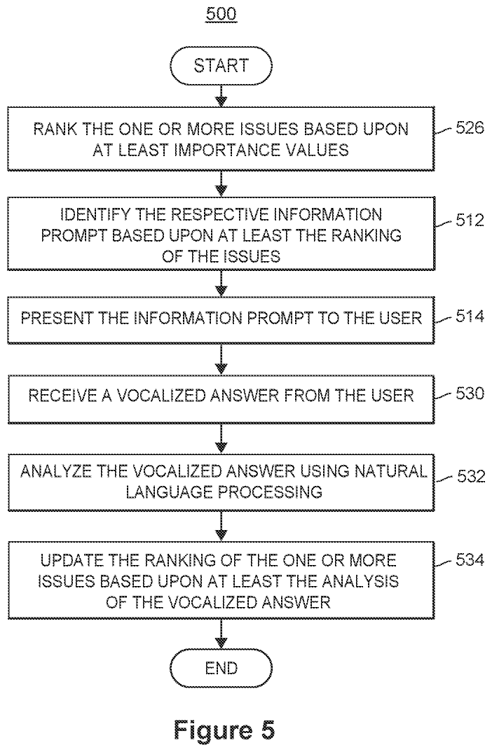

FIG. 5 illustrates a flow diagram of an exemplary computer-implemented method 500 for generating a user log containing a plurality of user data entries to indicate an exemplary flow for receiving a vocalized answer from a user and updating a ranking based upon at least an analysis of the vocalized answer. Any similar numbering between FIGS. 3A-4 and FIG. 5 indicates a similar action. The computer-implemented method 500 is exemplary only, and other methods may include additional, fewer, or alternative actions, including those discussed elsewhere herein.

Various parts of the computer-implemented guided user data collection and recommendation generation method 500 may be implemented by or performed using the various front-end components 2 and back-end components 4, which may communicate via the network 3, as described above. In some embodiments, the computer-implemented guided user data collection and recommendation generation method 500 may be implemented by one or more servers of a cloud computing service (e.g., one or more servers 40 configured to implement a single guided user data collection and recommendation generation platform).

At block 526, the conversational artificial intelligence model 54 ranks the one or more issues based upon at least importance values. The conversational artificial intelligence model 54 may be trained by processing large quantities of data via machine learning techniques. In some embodiments, the data may come from the user carrying out computer-implemented method 500. In further embodiments, the data may come from other users of the application. In still further embodiments, the data may come from a third party source. In some implementations, the conversational artificial intelligence model 54 is trained using deep learning and/or neural network techniques.

At block 512, the conversational artificial intelligence model 54 identifies the respective information prompt based upon at least the ranking of the issues. In some embodiments, the conversational artificial intelligence model 54 removes issues common to potential prompts when identifying the respective information prompt.

At block 514, the client device 110 presents the information prompt to the user. In some embodiments, the client device 110 may present the information prompt to the user visually via a display 112. In further embodiments, the client device 110 may present the information prompt to the user through an audio-based method, such as speakers. In still further embodiments, the client device 110 may present the information prompt to the user through some combination of visual and audio-based methods.

At block 530, the client device 110 receives a vocalized answer from the user via the input 114. In some embodiments, the input 114 may be a microphone embedded within the client device 110. In other embodiments, the input 114 may be an external microphone connected to the client device through an external port. The vocalized answer may be an indicated response included in the information prompt (e.g., “Is your temperature above 100.8 degrees Fahrenheit? Say ‘yes’ or ‘no’ now.”). In other implementations, the vocalized answer may be a lengthier, freeform response. For example, the user may respond to an information prompt of “Have you been drinking enough water?” with a response along the lines of “Well, I've had 2 cups today. No, make that 3—I forgot about the one after walking the dog.”

At block 532, the conversational artificial intelligence model 54 analyzes the vocalized answer using NLP techniques. In some embodiments, the conversational artificial intelligence model 54 may analyze the answer using NLP techniques in conjunction with machine learning techniques. In some implementations, the conversational artificial intelligence model 54 may identify keywords using NLP techniques and base the analysis at least in part upon the keywords. In other implementations, the conversational artificial intelligence model 54 may analyze the response in its entirety.

At block 534, the conversational artificial intelligence model 54 updates the ranking of the one or more issues based upon at least the analysis of the vocalized answer. In some embodiments, the presence and/or lack of certain keywords may cause the conversational artificial intelligence model 54 to modify the ranking of the one or more issues. In further embodiments, the conversational artificial intelligence model 54 may determine that certain issues are no longer relevant based upon the analysis of the vocalized answer. As such, the conversational artificial intelligence model 54 removes the irrelevant issues from the ranking. Similarly, in some embodiments the analysis of the vocalized answer may confirm and/or guarantee the presence of an issue. As such, the conversational artificial intelligence model 54 makes note of and removes the confirmed issues from the ranking.

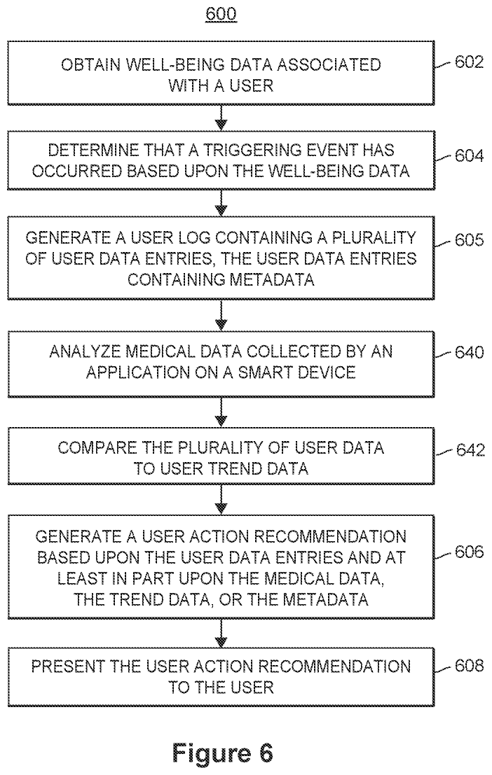

FIG. 6 illustrates a flow diagram of an exemplary computer-implemented method 600 for generating a user log containing a plurality of user data entries to indicate an exemplary flow using well-being data in determining a triggering event has occurred and using collected medical data, trend data, and metadata in generating a user action recommendation. Any similar numbering between FIGS. 3A-5 and FIG. 6 indicates a similar action. The computer-implemented method 600 is exemplary only, and other methods may include additional, fewer, or alternative actions, including those discussed elsewhere herein.

Various parts of the computer-implemented guided user data collection and recommendation generation method 600 may be implemented by or performed using the various front-end components 2 and back-end components 4, which may communicate via the network 3, as described above. In some embodiments, the computer-implemented guided user data collection and recommendation generation method 600 may be implemented by one or more servers of a cloud computing service (e.g., one or more servers 40 configured to implement a single guided user data collection and recommendation generation platform).

At block 602, the server 40 obtains well-being data associated with a user. In some embodiments, the well-being data includes at least one of (i) social media data, (ii) browsing data, (iii) biometric data, (iv) smart device data, (v) geolocation data, or (vi) user-input data. In some embodiments, the well-being data is gathered by a client device 110. In further embodiments, the well-being data is gathered from user data in a database 46 on server 40. In still further embodiments, the well-being data is gathered from a third party.

At block 604, the server 40 determines that a triggering event has occurred based upon the well-being data. The triggering event may be based upon any or all of the well-being data. For example, the server 40 may make use of geolocation and temporal data to recognize that a user has left the workplace earlier than usual, which may flag to the server 40 that a triggering event has occurred. Physical health data or scheduling data may indicate that a triggering event has not actually occurred despite the change to routine, however, and computer-implemented method 600 may cease.

At block 605, the server 40, using the conversational artificial intelligence model 54, generates a user log containing a plurality of user data entries, the user data entries containing metadata. In some embodiments, the metadata regards an aspect of the user response associated with at least one of: (i) a time, (ii) a length, (iii) a duration, or (iv) a delay of the user response. For example, in an instance in which metadata regarding the delay of the user response is included within the user data entry, the conversational artificial intelligence model 54 may make note that the user took a longer than average time to respond. The metadata, then, may potentially indicate a more serious outcome.

At block 640, the server 40 analyzes medical data collected by an application on a smart device. In some embodiments, the medical data is input by the user into an application. In further embodiments, the application accesses data input by another, such as a medical professional. In some implementations, the application that collects medical data is the same application through which computer-implemented method 600 is implemented.

At block 642, the server 40 compares the plurality of user data to user trend data. In some embodiments, user trend data includes past data collected from the user (e.g., past instances where the user has left work early, past instances where the user has felt lightheaded, past prescriptions from doctors, etc.). In further embodiments, user trend data includes user data collected from other users, with permission. The trend data collected from other users may include metadata, well-being data, or large-scale medical data. In still further embodiments, the trend data is collected from third party sources.

At block 606, the server 40 generates a user action recommendation based upon the user data entries and at least based in part upon the medical data, the trend data, and/or the metadata. For example, in an embodiment in which the user action recommendation is based upon the user data entries and the metadata, the recommendation may include calling for a ride to the hospital where the user takes too long to respond, potentially meaning that the user is dazed and/or confused. As another example, symptoms that may point to one cause may be redirected to another based upon the medical data of a user—a tendency for heart disease, for example. Similarly, trend data may indicate that a proper recommendation is to purchase an over-the-counter medicine from a pharmacy rather than get a prescription from a doctor.

At block 608, the client device 110 presents the user action recommendation to the user. In some embodiments, the server 40 may present the user action recommendation visually via a display 112 of a client device 110 associated with the user. In further embodiments, the server 40 may present the user action recommendation through an audio-based method, such as via speakers of the client device 110. In still further embodiments, the server 40 may present the user action recommendation to the user via some combination of visual and audio-based means.

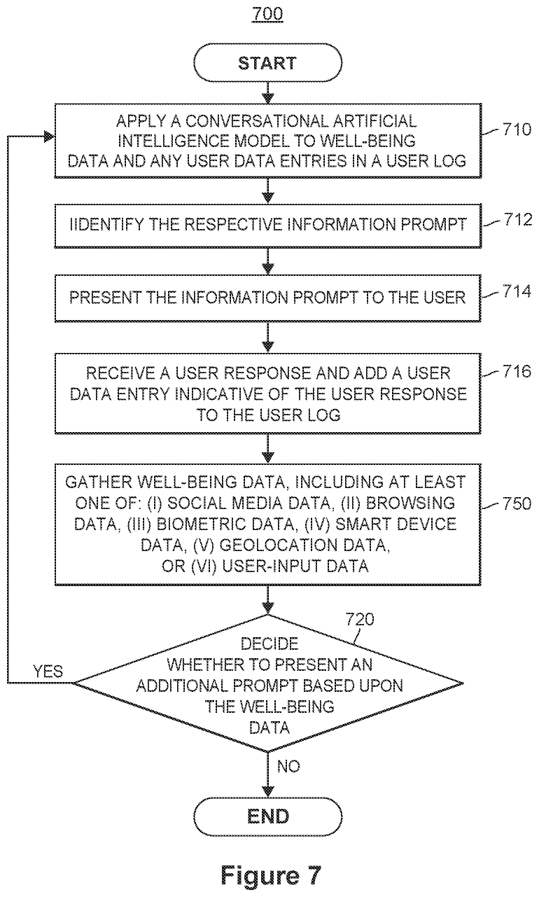

FIG. 7 illustrates a flow diagram of an exemplary computer-implemented method 700 for generating a user log containing a plurality of user data entries to indicate an exemplary flow for determining whether to present an additional prompt based upon at least well-being data. Any similar numbering between FIGS. 3A-6 and FIG. 7 indicates a similar action. The computer-implemented method 700 is exemplary only, and other methods may include additional, fewer, or alternative actions, including those discussed elsewhere herein.

Various parts of the computer-implemented guided user data collection and recommendation generation method 700 may be implemented by or performed using the various front-end components 2 and back-end components 4, which may communicate via the network 3, as described above. In some embodiments, the computer-implemented guided user data collection and recommendation generation method 700 may be implemented by one or more servers of a cloud computing service (e.g., one or more servers 40 configured to implement a single guided user data collection and recommendation generation platform).

At block 710, the server 40 applies a conversational artificial intelligence model 54 to well-being data and any user data entries in a user log. The conversational artificial intelligence model 54 may be trained by processing large quantities of data via machine learning techniques. In some embodiments, the data may come from the user carrying out computer-implemented method 700. In further embodiments, the data may come from other users of the application. In still further embodiments, the data may come from a third party source. In other implementations, the conversational artificial intelligence model 54 is trained using deep learning and/or neural network techniques.

At block 712, the conversational artificial intelligence model 54 identifies the respective information prompt. In some embodiments, this identification may be based at least in part upon a valuation and ranking system implemented by the conversational artificial intelligence model 54. In further embodiments, this identification may be based at least in part upon well-being data. The well-being data may include user data regarding physical health, mental health, social data, financial records, official records, account data, geolocation data, temporal data, etc.

At block 714, the conversational artificial intelligence model 54 presents the information prompt to the user via the client device 110. In some embodiments, the client device 110 may present the information prompt to the user visually via a display 112. In further embodiments, the client device 110 may present the information prompt to the user through an audio-based method, such as speakers. In still further embodiments, the client device 110 presents the information prompt to the user through some combination of visual and audio-based means.

At block 716, the client device 110 receives a user response and transmits the user response to the server 40. The server 40 adds a user data entry indicative of the user response to the user log via the conversational artificial intelligence model 54. In some embodiments, the conversational artificial intelligence model 54 uses NLP techniques to analyze the user response.

At block 750, the conversational artificial intelligence model 54 gathers well-being data, including at least one of (i) social media data, (ii) browsing data, (iii) biometric data, (iv) smart device data, (v) geolocation data, or (vi) user-input data. In some embodiments, the well-being data is gathered by a client device 110 and transmitted to the conversational artificial intelligence model 54. In further embodiments, the well-being data is gathered from user data in a database 46 on server 40. In still further embodiments, the well-being data is gathered from a third party.

At block 720, the conversational artificial intelligence model 54 decides whether to present an additional prompt based at least upon the well-being data. If so, the computer-implemented method loops back to block 710. If not, the computer-implemented method continues on. For example, the conversational artificial intelligence model 54 may use biometric data collected via a client device 110 and/or a wearable smart device to determine that a user response is incorrect. The conversational artificial intelligence model 54 may then decide to present an additional prompt based upon the collected biometric data. The conversational artificial intelligence model 54 is then applied to more well-being data and any user data entries in a user log.

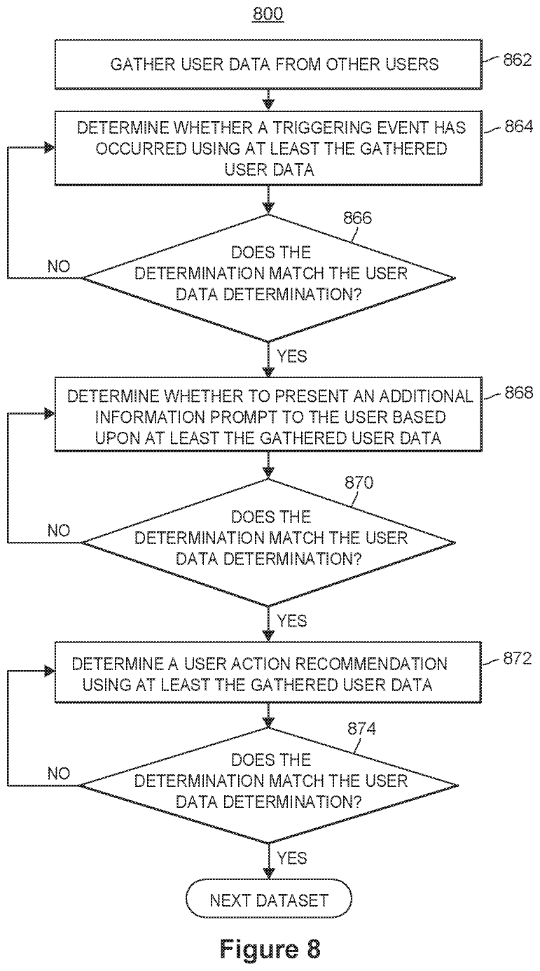

FIG. 8 illustrates a flow diagram of an exemplary computer-implemented method 800 for training a conversational artificial intelligence using gathered user data. Though the description below discusses mainly making use of user data from other users, the training may instead use data from third parties depending upon the embodiment. The computer-implemented method 800 is exemplary only, and other methods may include additional, fewer, or alternative actions, including those discussed elsewhere herein.

Various parts of the computer-implemented guided user data collection and recommendation generation method 800 may be implemented by or performed using the various front-end components 2 and back-end components 4, which may communicate via the network 3, as described above. In some embodiments, the computer-implemented method for training a conversational artificial intelligence 800 may be implemented by one or more servers of a cloud computing service (e.g., one or more servers 40 configured to implement a single guided user data collection and recommendation generation platform).

At block 862, the server 40 gathers user data from other users. In some embodiments, the gathered user data includes at least: (i) the raw data originally gathered from the users in question, (ii) the determination as to whether a triggering event occurred, (iii) the determination as to whether an additional information prompt was to be presented, and (iv) the user action recommendation.

At block 864, the conversational artificial intelligence model 54 determines whether a triggering event has occurred using at least the gathered user data. In some embodiments, the conversational artificial intelligence model 54 makes the determination using well-being data from the gathered user data. The well-being data may include user data regarding physical health, mental health, social data, financial records, official records, account data, geolocation data, temporal data, etc.

At block 868, the conversational artificial intelligence model 54 determines whether to present an additional information prompt to the user based upon at least the gathered user data. In some embodiments, the conversational artificial intelligence model 54 makes the determination using well-being data from the gathered user data. In further embodiments, the conversational artificial intelligence model 54 makes the determination based upon simulated and/or real user responses determined from and/or gathered from the user data.

At block 872, the conversational artificial intelligence model 54 determines a user action recommendation using at least the gathered user data. In some embodiments, the user action recommendation is based at least in part upon metadata included in the gathered user data. In further embodiments, the user action recommendation is based at least in part upon a determination included in the gathered user data to present an additional information prompt to the user. In still further embodiments, the user action recommendation is based at least in part upon the conversational artificial intelligence model 54 deciding to present an additional information prompt to the user. In some implementations, the conversational artificial intelligence model 54 simulates user responses and determines a user action recommendation based at least in part upon the simulated user responses.

At each of blocks 866, 870, and 874, the conversational artificial intelligence model 54 compares the determination made by the conversational artificial intelligence model 54 to the determination extant in the gathered user data. In instances where the two determinations match, the conversational artificial intelligence model 54 does not modify the algorithm used. In instances where the two determinations do not match, the conversational artificial intelligence model modifies the algorithm used by the conversational artificial intelligence model 54. The computer-implemented method 800 then loops to the previous block and the conversational artificial intelligence model 54 makes a new determination.

Though FIG. 8 and computer-implemented method 800 depict one potential order for the training, it should be noted that any order of block sets 864/866, 868/870, and/or 872/874 is also possible. In addition, training the conversational artificial intelligence model 54 using any of the block sets 864/866, 868/870, and/or 872/874 individually or in combination is possible.

### Other Matters

Although the preceding text sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

With the foregoing, an insurance customer may opt in to a program to receive a reward, insurance discount, or other type of benefit. In some aspects, customers may opt in to a rewards, loyalty, or other program associated with guided user data collection and recommendation generation, such as a rewards program that collects data regarding use of a guided user data collection program to determine discounts for appropriate use of generated recommendations. The customers may therefore allow a remote server to collect sensor, telematics, vehicle, mobile device, and other types of data discussed herein. With customer permission or affirmative consent, the data collected may be analyzed to provide certain benefits to customers. For instance, insurance cost savings may be provided to lower risk or risk averse customers. Recommendations that lower risk or provide cost savings to customers may also be generated and provided to customers based upon data analysis, as discussed elsewhere herein. Other functionality or benefits of the systems and methods discussed herein may also be provided to customers in return for them allowing collection and analysis of the types of data discussed herein. In return for providing access to data, risk-averse insureds and/or application users may receive discounts or insurance cost savings on life and health-related insurance, as well as home, renters, personal articles, transportation and other types of insurance from the insurance provider.

Throughout this specification, plural instances may implement components, operations, or structures described as a single instance. Although individual operations of one or more computer-implemented methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more modules of a computer system (e.g., a processor or a group of processors) may be configured by software (e.g., an application or application portion) as a module that operates to perform certain operations as described herein.

In various embodiments, a module may be implemented mechanically or electronically. For example, a module may comprise dedicated circuitry or logic that is permanently configured (e.g., as a special-purpose processor, such as a field programmable gate array (FPGA) or an application-specific integrated circuit (ASIC) to perform certain operations. A module may also comprise programmable logic or circuitry (e.g., as encompassed within a general-purpose processor or other programmable processor) that is temporarily configured by software to perform certain operations. It will be appreciated that the decision to implement a module mechanically, in dedicated and permanently configured circuitry, or in temporarily configured circuitry (e.g., configured by software) may be driven by cost and time considerations.

Accordingly, the term “module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules can provide information to, and receive information from, other modules. Accordingly, the described modules may be regarded as being communicatively coupled. Where multiple such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory device to which it is communicatively coupled. A further module may then, at a later time, access the memory device to retrieve and process the stored output. Modules may also initiate communications with input or output devices, and may operate on a resource (e.g., a collection of information).

The various operations of example computer-implemented methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the computer-implemented methods or routines described herein may be at least partially processor-implemented. For example, at least some of the operations of a computer-implemented method may be performed by one or more processors or processor-implemented modules. The performance of certain of the operations may be distributed among the one or more processors, not only residing within a single machine, but deployed across a number of machines. In some example embodiments, the one or more processors or processor-implemented modules may be located in a single geographic location (e.g., at a location of a mobile computing device or at a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information. Such memories may be or may include non-transitory, tangible computer-readable media configured to store computer-readable instructions that may be executed by one or more processors of one or more computer systems.

As used herein any reference to “one embodiment” or “an embodiment” means that a particular element, feature, structure, or characteristic described in connection with the embodiment is included in at least one embodiment. The appearances of the phrases “in one embodiment,” “in an embodiment,” “in some embodiments,” or similar phrases in various places in the specification are not necessarily all referring to the same embodiment or the same set of embodiments.

Some embodiments may be described using the terms “coupled,” “connected,” “communicatively connected,” or “communicatively coupled,” along with their derivatives. These terms may refer to a direct physical connection or to an indirect (physical or communicative) connection. For example, some embodiments may be described using the term “coupled” to indicate that two or more elements are in direct physical or electrical contact. The term “coupled,” however, may also mean that two or more elements are not in direct contact with each other, but yet still co-operate or interact with each other. Unless expressly stated or required by the context of their use, the embodiments are not limited to direct connection.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless the context clearly indicates otherwise.

This detailed description is to be construed as exemplary only and does not describe every possible embodiment, as describing every possible embodiment would be impractical, if not impossible. One could implement numerous alternate embodiments, using either current technology or technology developed after the filing date of this application.

Upon reading this disclosure, those of skill in the art will appreciate still additional alternative structural and functional designs for the systems and computer-implemented methods disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the computer-implemented method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

Finally, the patent claims at the end of this patent application are not intended to be construed under 35 U.S.C. § 112(f), unless traditional means-plus-function language is expressly recited, such as “means for” or “step for” language being explicitly recited in the claims. The systems and methods described herein are directed to an improvement to computer functionality, which may include improving the functioning of conventional computers in performing tasks.

What is claimed is:

1. A computer-implemented method for guided user data collection and recommendation generation, comprising:
obtaining, by one or more processors, well-being data associated with a user;
determining, by the one or more processors, occurrence of a triggering event associated with the user based upon the well-being data;
generating, by the one or more processors, a user log containing a plurality of user data entries by iteratively presenting a plurality of information prompts to the user in response to the occurrence of the triggering event by, for each of the plurality of information prompts:
applying a conversational artificial intelligence model to the well-being data and any user data entries in the user log to identify the respective information prompt;
presenting the information prompt to the user via a user interface;
receiving a user response to the information prompt via the user interface;
adding a user data entry indicative of the user response to the user log; and
determining whether to present an additional information prompt to the user;
generating, by the one or more processors, a user action recommendation based upon the user data entries of the user log; and
presenting, via the user interface, the user action recommendation to the user.

2. The computer-implemented method of claim 1, further comprising:
identifying, by the one or more processors, one or more issues associated with the triggering event;
generating, by the one or more processors, an importance value for each of the one or more issues associated with the triggering event;
ranking, by the one or more processors, the one or more issues based upon at least the importance values; and
identifying, by the one or more processors, the respective information prompt based upon at least the ranking of the one or more issues.

3. The computer-implemented method of claim 2, further comprising:
receiving, via the user interface, a vocalized answer from the user;
analyzing, by the one or more processors and using natural language processing, the vocalized answer; and
updating, by the one or more processors and based upon at least the analysis of the vocalized answer, the ranking of the one or more issues.

4. The computer-implemented method of claim 1, wherein generating the user action recommendation includes analyzing medical data collected by an application on a smart device.

5. The computer-implemented method of claim 1, wherein the well-being data includes at least one of: (i) social media data, (ii) browsing data, (iii) biometric data, (iv) smart device data, (v) geolocation data, or (vi) user-input data.

6. The computer-implemented method of claim 1, wherein the determination whether to present an additional information prompt to the user is based at least in part upon well-being data, including at least one of: (i) social media data, (ii) browsing data, (iii) biometric data, (iv) smart device data, (v) geolocation data, or (vi) user-input data.

7. The computer-implemented method of claim 1, wherein generating the user action recommendation includes comparing, by the processors, the plurality of user data to user trend data, the user trend data including at least one of: (i) collected user data from other users, (ii) past user data from the user, or (iii) user data from a third party database.

8. The computer-implemented method of claim 1, wherein:
one or more of the user data entries in the user log further contain metadata regarding an aspect of the user response associated with: (i) a time, (ii) a length, (iii) a duration, or (iv) a delay of the user response; and
the user action recommendation is based at least in part upon the metadata.

9. The computer-implemented method of claim 1, further comprising:
training, via a machine-learning algorithm, the conversational artificial intelligence model using at least one of: (i) collected user data from other users, (ii) past user data from the user, or (iii) user data from a third party database.

10. A computer system for guided user data collection and recommendation generation, comprising:
one or more processors;
a program memory coupled to the one or more processors and storing executable instructions that, when executed by the one or more processors, cause the computer system to:
obtain well-being data associated with a user;
determine occurrence of a triggering event associated with the user based upon the well-being data;
generate a user log containing a plurality of user data entries by iteratively presenting a plurality of information prompts to the user in response to the occurrence of the triggering event by, for each of the plurality of information prompts:
applying a conversational artificial intelligence model to the well-being data and any user data entries in the user log to identify the respective information prompt;
presenting the information prompt to the user via a user interface;
receiving a user response to the information prompt via the user interface;
adding a user data entry indicative of the user response to the user log; and
determining whether to present an additional information prompt to the user;
generate a user action recommendation based upon the user data entries of the user log; and
present the user action recommendation to the user.

11. The computer system of claim 10, wherein the executable instructions further cause the computer system to:
identify one or more issues associated with the triggering event;
generate an importance value for each of the one or more issues associated with the triggering event;
rank the one or more issues based upon at least the importance values; and
identify the respective information prompt based upon at least the ranking of the one or more issues.

12. The computer system of claim 11, wherein the executable instructions further cause the computer system to:
receive a vocalized answer from the user;
analyze, using natural language processing, the vocalized answer; and
update, based upon at least the analysis of the vocalized answer, the ranking of the one or more issues.

13. The computer system of claim 10, wherein the executable instructions that cause the computer system to generate the user action recommendation cause the computer system to analyze medical data collected by an application on a smart device.

14. The computer system of claim 10, wherein the well-being data includes at least one of: (i) social media data, (ii) browsing data, (iii) biometric data, (iv) smart device data, (v) geolocation data, or (vi) user-input data.

15. The computer system of claim 10, wherein the executable instructions that cause the computer system to determine whether to present an additional information prompt to the user cause the computer system to make such determination based at least in part upon well-being data, including at least one of: (i) social media data, (ii) browsing data, (iii) biometric data, (iv) smart device data, (v) geolocation data, or (vi) user-input data.

16. The computer system of claim 10, wherein the executable instructions that cause the computer system to generate the user action recommendation cause the computer system to compare the plurality of user data to user trend data, the user trend data including at least one of: (i) collected user data from other users, (ii) past user data from the user, or (iii) user data from a third party database.

17. The computer system of claim 10, wherein:
one or more of the user data entries in the user log further contain metadata regarding an aspect of the user response associated with: (i) a time, (ii) a length, (iii) a duration, or (iv) a delay of the user response; and
the user action recommendation is based at least in part upon the metadata.

18. The computer system of claim 10, wherein the executable instructions further cause the computer system to:
train, via a machine-learning algorithm, the conversational artificial intelligence model using at least one of: (i) collected user data from other users, (ii) past user data from the user, or (iii) user data from a third party database.

19. A non-transitory computer-readable medium storing instructions for guided user data collection and recommendation generation that, when executed by one or more processors of a computer system, cause the computer system to:
obtain well-being data associated with a user;
determine occurrence of a triggering event associated with the user based upon the well-being data;
generate a user log containing a plurality of user data entries by iteratively presenting a plurality of information prompts to the user in response to the occurrence of the triggering event by, for each of the plurality of information prompts:
applying a conversational artificial intelligence model to the well-being data and any user data entries in the user log to identify the respective information prompt;
presenting the information prompt to the user via a user interface;
receiving a user response to the information prompt via the user interface;
adding a user data entry indicative of the user response to the user log; and
determining whether to present an additional information prompt to the user;
generate a user action recommendation based upon the user data entries of the user log; and
present the user action recommendation to the user.

20. The non-transitory computer-readable medium of claim 19, further storing instructions that, when executed by the one or more processors, cause the computer system to:
identify one or more issues associated with the triggering event;
generate an importance value for each of the one or more issues associated with the triggering event;
rank the one or more issues based upon at least the importance values; and
identify the respective information prompt based upon at least the ranking of the one or more issues.
